(19) United States
(12) Patent Application Publication
(10) Pub. No.: US 2024/0203542 A1
(43) Pub. Date: Jun. 20, 2024

(54) SELF-SAMPLING MANAGEMENT ELECTRONIC DEVICE, SELF-SAMPLING MANAGEMENT METHOD, AND COMPUTER READABLE MEDIUM STORING PROGRAM TO PERFORM THE METHOD

(71) Applicant: SEEGENE, INC., Seoul (KR)

(72) Inventors: Young Sahng SUH, Seoul (KR); Young Wook KIM, Seoul (KR)

(21) Appl. No.: 18/287,911

(22) PCT Filed: Apr. 22, 2022
(86) PCT No.: PCT/KR2022/005784

§ 371 Date: Oct. 23, 2023

(30) Foreign Application Priority Data
Apr. 22, 2021 (KR) 10-2021-0052579

(51) Int. Cl. G16H 10/40; G06T 7/20; G06T 7/70; G06V 40/10; G06V 40/20

(52) U.S. Cl. CPC G16H 10/40; G06T 7/20; G06T 7/70; G06V 40/10; G06V 40/20; G06T 2207/30004; G06T 2207/30201

(57) ABSTRACT

A self-sampling management electronic device includes a memory stores instructions and at least one processor. As the instructions are executed, the processor tracks a sampling action of a specimen provider using images of the specimen provider handling a specimen sampling kit. The processor also verifies whether or not the specimen provider is the same person as a person who has requested the specimen sampling kit, by using at least one of the imaged used for the tracking.

### TECHNICAL FIELD

The present disclosure relates to a self-sampling management electronic device, a self-sampling management method, and a computer readable medium storing a program to perform the method

### BACKGROUND ART

Recently, severe acute respiratory syndrome coronavirus 2 (SARS-COV-2), a type of respiratory infection, has been pandemic. The fatality rate of SARS-COV-2 is relatively high among children, elderly people, people with weakened immunity, or patients with underlying conditions.

In most contagious diseases including SARS-COV-2, it is important to identify a causative pathogen, so that the contagious pattern can be accurately identified and effectively controlled.

To date, a variety of diagnostic approaches for identifying causative pathogens have been studied and developed. Molecular diagnostics is one of such approaches. In such molecular diagnostics, real-time polymerase chain reaction (PCR) is an approach able to rapidly and accurately detect a causative pathogen, as compared to other diagnostic techniques.

Real-time PCR includes a process of sampling a specimen from a person who has requested such a diagnosis. At present, individuals who perform such sampling are health professionals in most cases, but this may not always be the case. For example, in a situation in which it is difficult for the person to visit a specimen sampling location, a person may sample his or her own specimen (i.e., perform self-sampling) by receiving a specimen sampling kit necessary for sampling via mail or the like. Alternatively, a carrier who has delivered a specimen sampling kit to a person may collect the specimen from the person.

### DISCLOSURE OF INVENTION

### Technical Problem

A specimen should be sampled from a person who has requested a diagnosis.

However, in some cases, a specimen may be sampled from a third person instead of from the person. For example, a person afraid of being definitively diagnosed with a contagious disease may ask a third person that the specimen be sampled from the third person instead of from him or herself. In particular, in the case of self-sampling performed by the person or sampling performed on the person by a carrier, such a situation may occur.

In such a case, it may be impossible to accurately determine who is infected by the contagious disease, and thus anti-epidemic measures may be ineffective.

Accordingly, an objective to be solved according to an embodiment is to provide a technological solution for verifying whether or not a specimen provider, i.e., a person who has provided a specimen in a sampling process, is the same person as a person.

In addition, it is most effective to verify whether or not the sampling has been properly performed in the sampling process. To enable evaluation or feedback regarding actions related to the sampling to be delivered to a subject performing the sampling or a diagnosis center managing the sampling may be included in the objective.

### Solution to Problem

A self-sampling management electronic device according to an embodiment may include: a memory storing instructions; and at least one processor. The processor may be configured, by executing the instructions, to: track a sampling action of a specimen provider using images of the specimen provider handling a specimen sampling kit; and
verify whether or not the specimen provider is the same person as a person who has requested the specimen sampling kit, by using at least one image among the images used for the tracking.

The processor may be configured, by executing the instructions, to: compare sampling action information obtained from the tracking with reference sampling action information to be followed by the specimen provider when the specimen performs the sampling action; and
generate contents to be provided to the specimen provider on basis of a result of the comparison.

The reference sampling action information may include information regarding a reference depth of insertion and a reference angle of insertion required when a swab of the specimen sampling kit is inserted into a nose or a mouth. The sampling action information may include information regarding a measured depth of insertion and a measured angle of insertion measured from an image of the swab inserted into the nose or the mouth of the specimen provider from the image of the specimen provider handling a specimen sampling kit.

The tracked sampling action may include at least one of: a first action of unpacking the specimen sampling kit; a second action of grasping a swab included in the unpacked specimen sampling kit by the specimen provider or a third person; a third action of inserting the grasped swab into the nose or the mouth of the specimen provider; a fourth action of taking the swab out from the nose or the mouth of the specimen provider; a fifth action of putting the swab into a receiving vessel included in the specimen sampling kit; and a sixth action of sealing the receiving vessel in which the swab is received.

The processor may be configured, by executing the instructions, to: verify whether or not the tracked sampling action includes the first to sixth actions; and generate contents to be provided to the specimen provider on basis of a result of the evaluation.

The image used in the verification may be an image captured while one action among the first to sixth actions is being performed.

The selected one action may include the third action.

The selected one action may be randomly selected from the first to sixth actions.

In addition, when the specimen provider is not the same person as the person, the processor may generate a message to be provided to the specimen provider or a message to be transmitted to a terminal of a diagnosis center managing the specimen sampling kit.

A self-sampling management method according to an embodiment performed by a self-sampling management electronic device may include: tracking a sampling action of a specimen provider from an image of the specimen provider handling a specimen sampling kit; and verifying whether or not the specimen provider is the same person as a person who has requested the specimen sampling kit on basis of at least a portion of an image captured after start and before end of the tracking among the image of the specimen provider handling the specimen sampling kit.

A computer program stored in a computer readable recording medium according to an embodiment may be programmed to perform the operations included in the above-described self-sampling management method.

A computer readable recording medium according to an embodiment may include a computer program configured to perform the actions included in the above-described self-sampling management method.

An input/output (I/O) terminal according to an embodiment include: an image-capturing part, a communication part, and a display part. The image-capturing part may acquire an image of a specimen provider handling a specimen sampling kit by capturing the image. The communication part may transmit the acquired image to the electronic device and, responsively, receive a result of verification as to whether the specimen provider is the same person as the person who requested the specimen sampling kit. The display part may display the result of the verification received.

### Advantageous Effects of Invention

According to an embodiment, whether or not the specimen provider, i.e., the person who provided the specimen in the sampling process, is the same person as the person may be verified.

In addition, the verification or feedback regarding the actions related to the sampling may be delivered to the subject performing the sampling or the diagnosis center managing the sampling, and thus whether or not the sampling is properly performed may be verified.

### BRIEF DESCRIPTION OF DRAWINGS

FIG. 1 illustrates an example situation in which a self-sampling management electronic device according to an embodiment outputs a result of verification of sampling;

FIG. 2 illustrates an example situation in which information is delivered between a central management server, a diagnosis center, a treatment organization, and the self-sampling management electronic device according to an embodiment through a network;

FIG. 3 is a block diagram illustrating a configuration of the self-sampling management electronic device according to an embodiment;

FIG. 4 is a block diagram illustrating a computer program according to an embodiment, in which specific functions of the computer program are implemented as modules;

FIGS. 5 to 7 are diagrams illustrating examples that may be used for action tracking;

FIG. 8 is a flowchart illustrating a process in which the self-sampling management electronic device verifies whether or not the specimen provider is the same person as the person using the identity verification portion according to an embodiment;

FIG. 9 is a diagram schematically illustrating points in time at which the image of specimen provider is captured;

FIG. 10 is a diagram schematically illustrating the sampling result verification portion and input and output actions thereof;

FIG. 11 is a flowchart illustrating procedures of a self-sampling management method according to an embodiment;

FIG. 12 is a flowchart illustrating an information delivery process between the user terminal and the self-sampling management electronic device when the self-sampling management electronic device is implemented as a separate server;

FIG. 13 is a block diagram schematically illustrating a configuration of a self-sampling management electronic device according to another embodiment; and

FIG. 13 is a block diagram schematically illustrating a configuration of an I/O device according to another embodiment.

### BEST MODE FOR CARRYING OUT THE INVENTION

The advantages, features, and methods for realizing the same of the present disclosure will be more clearly understood from the following embodiments described hereinafter in detail with reference to the accompanying drawings. However, the present disclosure should not be construed as being limited to the embodiments set forth herein and may be embodied in a variety of different forms. Rather, these embodiments are provided so that the present disclosure will be thorough and complete, and will fully convey the scope of the present disclosure to those having ordinary knowledge in the technical field. The scope of the present disclosure shall be defined by the appended Claims.

Further, in the following description of embodiments of the present disclosure, detailed descriptions of well-known functions and components incorporated herein will be omitted when it is determined that the description may make the subject matter in some embodiments of the present invention rather unclear. In addition, terms used herein are defined in consideration of functions thereof in embodiments of the present disclosure, but may vary depending on the intentions of users or operators, as well as in practices. Therefore, the terms shall be defined on the basis of the description throughout the specification.

In the meantime, the terms' “part” or “portion” used herein may include software, hardware, or a combination thereof. Among such terms, software may be a machine language, firmware, embedded codes, or application software, or may be a model trained using a machine learning method. In some embodiments, a plurality of “parts” or “portions” may be embodied as a single unit or element, or a single “part” or “portion” may include a plurality of units or elements.

Hereinafter, examples of a person or a user may include a person (hereinafter, referred to as a “suspected patient”) suspected of being infected by at least one disease among a variety of diseases including a respiratory disease or a person (hereinafter, referred to as a “fully recovered person”) who has fully recovered from infection. In addition, the person or the user may include a common person not suspected of being infected by at least one disease among the above-described diseases. The person or the user may request that a diagnosis center, a central management server, or a treatment organization perform sampling on his or her specimen.

The diagnosis center may include a screening center. In the diagnosis center, a test is performed. The test includes in vitro diagnostics. The test may specifically include molecular diagnostics and immunodiagnostics, but is not limited thereto. A result of the test includes a diagnostics result regarding whether or not the person is infected by at least one of the above-described diseases. The diagnosis center may be provided with a terminal.

The treatment organization includes a medical organization, such as a hospital or a public health center. In the treatment organization, treatment of patients infected by at least one of the above-described diseases is performed. The treatment organization may be provided with a terminal.

The central management server collects test results from a plurality of persons of the diagnosis center and manages the collected test results.

Described in more detail, the central management server receives results of the test of a specific disease performed on a person, i.e., whether the person has tested positive or negative for the specific disease. On the basis of the test result received in this manner, the central management server may create an infection map. The position of a person who has requested a test and a test result for the person may be disposed on the infection map. In addition, the position of a person who has requested test, which has not yet been completed, may be disposed on the infection map.

In addition, the central management server may perform different subsequent actions depending on whether the test result by the diagnosis center is positive or negative.

For example, when the test result is positive, the central management server may request that the corresponding person be treated while transferring information regarding the corresponding person to the terminal of the treatment organization or the treatment organization.

In addition, after positional information is obtained from a mobile terminal of a person determined to be positive, the central management server may generate information regarding a prospective contact who is expected to have been in contact with the person. The information regarding the prospective contact may be generated, for example, on the basis of information regarding the owner of a mobile terminal that has accessed a mobile communication base station (hereinafter, referred to as a “base station”) the same as a base station that the mobile terminal of the person accessed. Alternatively, the information regarding the prospective contact may be generated on the basis of information regarding the access of the mobile terminal of the person to another terminal by a communication method, such as Bluetooth or near-field communication (NFC).

In addition, the central management server may request the prospective contact be tested, on the basis of the above-described information regarding the prospective contact. This request for the test may be transmitted to not only the mobile terminal of the prospective contact, but also the diagnosis center.

In addition, the central management server may allow the information regarding the prospective contact to be provided on the infection map.

In addition, the central management server may calculate the reproduction number R and display the reproduction number R on the infection map. The reproduction number R is defined by the average number of infected persons generated by a single infectious patient within a group. R indicates a value equal to or greater than 0. Here, R>1 means that at least one person may be additionally infected, in case of which the contagious disease may spread in the population group so that the epidemic will continue. In contrast, R<1 means that the epidemic is slowing and the number of persons infected by the contagious disease may be reduced. R=1 means continuous occurrence, and an endemic corresponds to continuation of this number.

The reproduction number R may be expressed by the equation: R=pXcXd. Here, p indicates a probability of infection, which may be reduced by medication, the use of masks, or the like. Further, c indicates a contact rate, the value of which may be reduced by strengthened social distancing, while d indicates a duration during which the infection spreads. The duration d may be reduced by rapid isolation of patients. Thus, governments are trying to prevent the spread of the contagious disease by anti-epidemic measures, such as the use of masks, social distancing, and rapid diagnostic tests. Accordingly, the calculation of Rt is an important indicator for evaluating the effect of the anti-epidemic policy and monitoring the aspect of the spread of infection.

The reproduction number may be categorized into a basic reproduction number RO, an effective reproduction number Re, a time-varying reproduction number Rt, and the like, which have the following characteristics, respectively.

The basic reproduction number RO means a reproduction number when a contagious disease has entered a population group not immune to the contagious disease (a novel contagious disease in an unvaccinated population), in which management of and intervention to stem the spread of the contagious disease have not occurred. The basic reproduction number RO is an indicator of biological infectivity, and is mainly calculated in the early stage of an epidemic. It is known that RO exhibits a similar range of values for generally the same contagious diseases, although RO is not a unique number assigned to each contagious disease.

The effective reproduction number Re means a reproduction number when an epidemic is ongoing after the early pattern of occurrence of a contagious disease, in consideration of personal hygiene management (e.g., intensified hygiene and social distancing) and social measures (e.g., anti-epidemic control, blockage, school restrictions, and bans on large gatherings). The reproduction number after the early stage of the epidemic of the contagious disease substantially refers to the effective reproduction number Re. The effective reproduction number Re is used to track changes in infectivity over time and review short-term effects of intervention.

A time-varying reproduction number Rt in the effective reproduction number Re is defined as average infectivity of a population group at a specific point in time, which may be calculated repeatedly over time. At a time t, Rt may be calculated by two methods of an instantaneous reproduction number and a case reproduction number. The instantaneous reproduction number is obtained by measuring the spread at a specific point in time, whereas the case reproduction number is obtained by measuring the spread by individuals of a specific cohort group. The case reproduction number is available for retrospective analysis regarding the manner in which individuals infected at different points in time have contributed to the spread (see “The Concept of Re production Number and Changes According to Government Response Policies”; Myeong-Su Yoo, Yeon-Ju Kim, Soo-Jin Back, and Dong-Hyok Kwon; Weekly health and diseases, 14 (6), Feb. 4, 2021).

The central management server may reflect the above-described information regarding the prospective contact in the calculation of the above-described reproduction number R.

Meanwhile, the above-described person or the user may request the national diagnosis center, the central management server, or the like deliver the specimen sampling kit to the person or the user. Then, the specimen sampling kit may be delivered to the person from the diagnosis center. In addition, the specimen sampling kit containing the specimen of the person may be collected by the diagnosis center. The diagnosis center may perform a test after acquiring the specimen from the collected specimen sampling kit.

Here, the specimen sampling kit refers to a packaged tool including a swab used in the sampling of the specimen and a receiving vessel for receiving the sampled specimen. In addition, the specimen sampling kit may further include at least one of a medium storage means including a specimen carrying medium and a sealing means for scaling the receiving vessel, in addition to the swab and the receiving vessel described above.

Among the above components, the swab may be implemented as a cotton swab, a syringe, a pipette, and the like, but is not limited thereto.

Meanwhile, the specimen subject to the sampling may be obtained from humans or animals, and examples thereof may include a resultant matter obtained by washing out the mouth, saliva, sputum, an oral swab, a nasal swab, blood, urine, stool, and the like, but are not limited thereto.

### MODE FOR THE INVENTION

Hereinafter, a variety of embodiments of the present disclosure will be described with reference to the drawings.

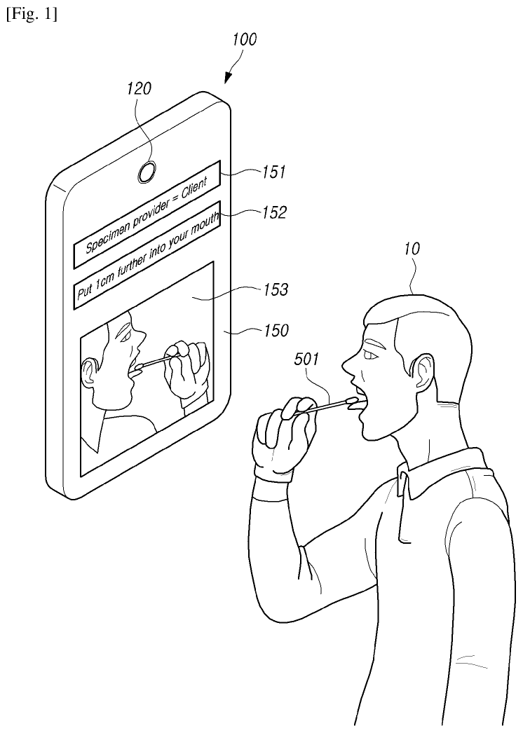

FIG. 1 illustrates an example situation in which a self-sampling management electronic device 100 according to an embodiment outputs a result of verification of sampling.

Referring to FIG. 1, a specimen provider 10 is sampling his own specimen from the throat using a swab 501 included in a specimen sampling kit. That is, the specimen provider 10 is performing self-sampling.

Furthermore, a display part 150 of the self-sampling management electronic device 100 is displaying a message 151 regarding a verification result as to whether or not the specimen provider 10 is the same person as a person. (The display part 150 will be described later in conjunction with FIG. 3.)

When the specimen provider 10 is verified to be the same person as the person, a message 151 regarding the verification is not only displayed on the display part 150 of the self-sampling management electronic device 100 but may also be transferred to the diagnosis center or the central management server to be described later.

In addition, the display part 150 of the self-sampling management electronic device 100 is displaying a verification result 152 as to whether or not the specimen provider 10 is properly performing a sampling action and a captured image 153 of the sampling action of the specimen provider 10. When the sampling action is verified to have been improperly performed, a message 152 regarding the improper sampling action is not only displayed on the display part 150 of the self-sampling management electronic device 100 but also may be transferred to the central management server.

Here, the captured image 153 is an image captured by an image-capturing part 120 of the self-sampling management electronic device 100. (The image-capturing part 120 will be described later in conjunction with FIG. 3.) Furthermore, each of the verification results and the messages 151 and 152 is a result verified on the basis of the captured image 153.

That is, according to an embodiment, whether or not a person who provided a specimen during a sampling process, i.e., the specimen provider 10, is the same person as the person may be verified by the self-sampling management electronic device 100. In addition, the self-sampling management electronic device 100 may transfer an evaluation or a feedback regarding actions related to the sampling to a person performing the sampling or the diagnosis center managing the sampling. In this manner, whether or not the sampling has been properly performed may be verified. Hereinafter, the self-sampling management electronic device 100 will be described in more detail.

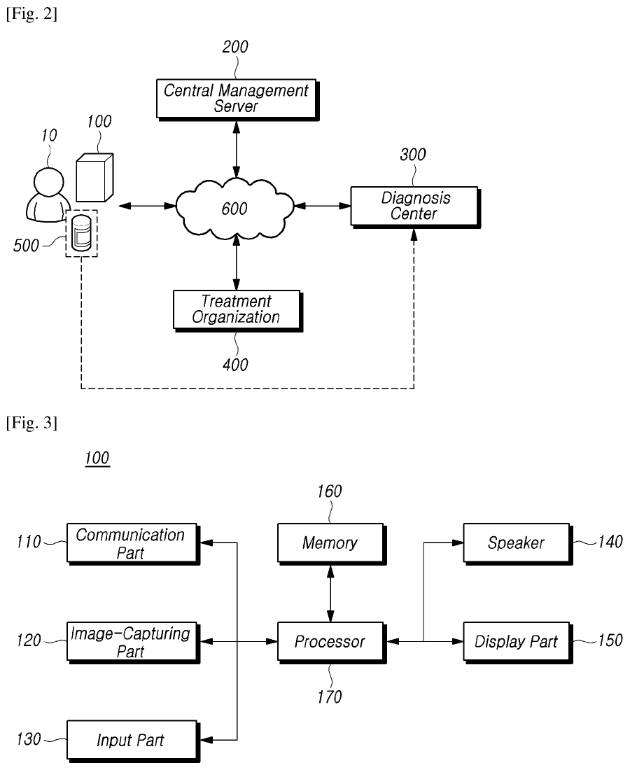

FIG. 2 illustrates an example situation in which information is delivered between a central management server 200, a diagnosis center 300, a treatment organization 400, and the self-sampling management electronic device 100 according to an embodiment through a network 600. Here, network 600 refers to a known wired network or a known wireless network.

Referring to FIG. 2, the specimen provider 10 possesses a specimen sampling kit 500 and the self-sampling management electronic device 100. That is, in FIG. 2, it is assumed that the specimen provider 10 performs self-sampling using the specimen sampling kit 500 or that a carrier (or delivery person) who has delivered the specimen sampling kit 500 to the specimen provider 10 performs sampling on the specimen provider 10. It is also assumed that the self-sampling management electronic device 100 is used in the sampling process. Here, a space in which the self-sampling is performed or a space in which the sampling is performed by the carrier may be a place, such as the house or the office of the specimen provider 10, an airport, a port, or a station, but is not limited thereto.

Hereinafter, the self-sampling or the sampling performed by the carrier will be described in more detail.

In the self-sampling, the specimen provider 10 samples his or her specimen by him or herself. In contrast, in the sampling performed by the carrier, the carrier samples the specimen from the specimen provider 10.

A body portion from which the specimen is sampled in any type of sampling may be the oral cavity, the anterior nasal cavity, or the like, but is not limited thereto. For example, sampling using a nasopharyngeal swab method is also possible. When the specimen is saliva, the specimen provider 10 may perform the sampling by inputting the swab 501 into his or her oral cavity or sample saliva spit by him or herself. In some cases, the specimen provider 10 may sample saliva spit by him or herself by inputting the swab 501 into his or her oral cavity, or perform the sampling in a reverse order.

When the sampling is completed, the specimen sampling kit 500 is delivered from the specimen provider 10 to the diagnosis center 300, as indicated with dotted lines in FIG. 2. Then, the specimen provider 10 may review information that his or her own specimen sampling kit 500 is delivered to the diagnosis center 300 through the self-sampling management electronic device 100. In the diagnosis center 300, a process of acquiring the specimen from the delivered specimen sampling kit 500 and a test process on the basis of the acquired specimen are performed.

The test result deduced in the diagnosis center 300 is delivered from the diagnosis center 300 or the terminal provided in the diagnosis center 300 to the self-sampling management electronic device 100. In addition, the test result may be delivered from the diagnosis center 300 or the terminal of the diagnosis center 300 to the central management server 200. Here, the central management server 200 collects and manages test results on a plurality of specimen providers including the specimen provider 10. The test results collected and managed in this manner may be used as source data for anti-epidemic measures.

Here, the above-described test result may be delivered within 24 hours or in next day from when the test was requested, or within several days.

In addition, the specimen provider 10 may be provided with the infection map including not only his or her own test result but also test results and positional information regarding other persons from the self-sampling management electronic device 100. Here, in some embodiments, the infection map may include positional information regarding another person whose test result has not yet been output even though a test has been requested.

In addition, when the specimen provider 10 is determined to be infected by a specific disease, i.e., has tested positive, on the basis of the test result, the test result and information regarding the specimen provider 10 are delivered to the treatment organization 400 or the terminal of the treatment organization 400. Then, the treatment organization 400 performs predetermined measures so that a treatment for the specimen provider 10 may be performed. Such measures for the specimen provider 10 include at least one of self-quarantine, quarantine in specific care facilities, and transfer to the treatment organization 400, but is not limited thereto.

When the specimen provider 10 is determined to have fully recovered from the specific disease through such measures, the specimen provider 10 may set his or her health status information (e.g., detailed questions about his or her condition) and positional information to be periodically delivered to the central management server 200 through the self-sampling management electronic device 100 for a predetermined period. Afterwards, when the predetermined period has passed, neither the health status information nor the positional information is delivered to the central management server 200 any further.

Differently, the specimen provider 10 may be determined to not be infected by the specific disease, i.e., to have tested negative, on the basis of the above test result. According to an embodiment, even in such a case, the specimen provider 10 may set his or her health status information (e.g., detailed questions about his or her condition) and positional information to be periodically delivered to the central management server 200 through the self-sampling management electronic device 100 for a predetermined period. Afterwards, when the predetermined period has passed, neither the health status information nor the positional information is delivered to the central management server 200 any further.

In addition, the self-sampling or the sampling by the carrier as described above may have some problems, which will be described as follows.

First, the specimen provider, i.e., a person who actually provided the specimen to the specimen sampling kit 500, may be a third person, even in the case that the specimen provider should be the person. For example, a person afraid of being definitively diagnosed with a contagious disease may ask a third person that the specimen be sampled from the third person instead of from him or herself.

In such a case, it may be impossible to accurately determine who is infected by the contagious disease, and thus anti-epidemic measures may be ineffective.

Second, the self-sampling or the sampling performed by the carrier may be inaccurate as compared to sampling performed by health professionals. Specifically described, a proper amount of specimen should be swabbed for an accurate diagnosis. In this regard, the swab should be inserted into a body portion, such as the oral cavity or the anterior nasal cavity, at a predetermined angle and to a predetermined depth. However, it may be difficult for a person who is not a health professional to insert the swab into the body portion at the set angle or to the set depth, as compared to health professionals. In particular, when the person is an elderly person or a visually impaired person, it would be more difficult.

Accordingly, the self-sampling management electronic device 100 according to an embodiment may be used to solve a variety of possible problems including the above-described two problems. The self-sampling management electronic device 100 will be described hereinafter.

FIG. 3 is a block diagram illustrating a configuration of the self-sampling management electronic device 100 according to an embodiment. Before the description of FIG. 3, the self-sampling management electronic device 100 will be described first.

The self-sampling management electronic device 100 may be various types of electronic devices.

As a first example, the self-sampling management electronic device 100 may be a portable communication terminal, a smartphone, a wearable device, a tablet PC, a desktop PC, a laptop computer, or the like.

As a second example, the self-sampling management electronic device 100 may be a kiosk or the like. In this case, the self-sampling management electronic device 100 is positioned in a public place, such as a station or an airport. In addition, the specimen provider 10 may be required to visit such a public place to use the self-sampling management electronic device 100.

A computer program may be operated in the self-sampling management electronic device 100. The computer program may include an application installed in the self-sampling management electronic device 100 during the fabrication thereof, or alternatively, an application downloaded from an application market or a server and installed in the self-sampling management electronic device 100 after the fabrication thereof.

When the computer program is executed by the self-sampling management electronic device 100, instructions in the computer program may be executed, thereby enabling a variety of processes to be performed by the self-sampling management electronic device 100. For example, a self-sampling management method according to an embodiment (to be described later in conjunction with FIG. 12) may be performed by the self-sampling management electronic device 100 in response to the execution of the computer program.

From now on, respective components of the self-sampling management electronic device 100 will be described.

Referring to FIG. 3, the self-sampling management electronic device 100 includes a communication part 110, the image-capturing part 120, an input part 130, a speaker 140, a display part 150, a memory 160, and a processor 170, but is not limited thereto. For example, the self-sampling management electronic device 100 may not include at least one of the components illustrated in FIG. 3 or further include any component not illustrated in FIG. 3.

The communication part 110 includes a wireless communication module. The wireless communication module may include at least one of long-term evolution (LTE), LTE advance (LTE-A), code division multiple access (CDMA), wideband CDMA (WCDMA), universal mobile telecommunications system (UMTS), wireless broadband (WiBro), wireless fidelity (WiFi), Bluetooth, near-field communication (NFC), and global navigation satellite system (GNSS), but is not limited thereto. The self-sampling management electronic device 100 may perform communications with entities 200, 300, and 400 illustrated in FIG. 2 or a variety of other entities not illustrated in FIG. 1 through the communication part 110.

The input part 130 includes an input module receiving predetermined information. Examples of the input module may include a keypad, a mouse, a keyboard, a touchscreen, buttons, and the like, but are not limited thereto.

The image-capturing part 120 is configured to generate an electric image signal by photoelectrically converting incident light. The generated image signal may be a still image or a video image.

The image-capturing part 120 may include not only an RGB camera but also a module capable of obtaining depth information, such as an infrared (IR) camera, a depth sensor, or a kinetic sensor, but is not limited thereto.

The speaker 140 is configured to output audio.

The display part 150 is configured to display various information. The display part 150 may include a panel, such as a liquid crystal display (LCD) or a light-emitting diode (LED) panel, but is not limited thereto.

The memory 160 stores instructions or data. In addition, a computer program, such as the above-described application, may be stored in the memory 160. The memory 160 may include a random access memory (RAM), a read only memory (ROM), a flash memory, and the like.

The processor 170 is configured to control the overall action of the self-sampling management electronic device 100. The processor 170 may be implemented as one or more processors.

The processor 170 may execute designated instructions, data, or an application. Thus, a variety of functions may be performed. Hereinafter, the computer program executable by the processor 170 according to an embodiment will be described.

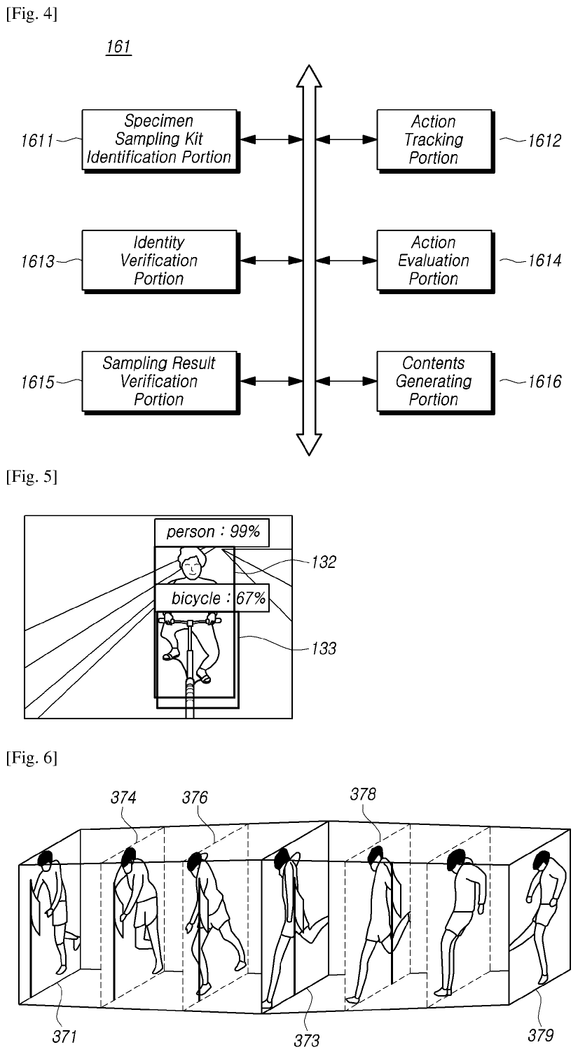

FIG. 4 is a block diagram illustrating a computer program 161 according to an embodiment, in which specific functions of the computer program 161 are implemented as modules. However, the block diagram illustrated in FIG. 4 is merely an example, and the idea of the present disclosure will not be interpreted as being limited to the block diagram illustrated in FIG. 4.

Referring to FIG. 4, the computer program 161 includes a specimen sampling kit identification portion 1611, an action tracking portion 1612, an identity verification portion 1613, an action evaluation portion 1614, a sampling result verification portion 1615, and contents generating portion 1616, but is not limited thereto.

The specimen sampling kit identification portion 1611 is configured to identify the specimen sampling kit 500.

In this regard, the specimen sampling kit identification portion 1611 may receive a captured image (i.e., a still image or a video image) regarding the specimen sampling kit 500 from the image-capturing part 120. Then, the specimen sampling kit identification portion 1611 identifies a unique ID assigned to the specimen sampling kit 500 or personal information regarding the person, i.e., a person who should provide a specimen to the specimen sampling kit, from the still image or the video image delivered.

Here, the still image or the video image delivered may include an image of a bar code or a QR code attached to the specimen sampling kit 500. In addition, the specimen sampling kit identification portion 1611 has a function of reading the bar code or the QR code appearing in the still image or the video image and acquiring the unique ID or the personal information (e.g., name, gender, or address) regarding the person from the bar code or the QR code. Here, the function of reading the bar code or the QR code appearing in the still image or the video image and acquiring the unique ID or the personal information regarding the person is a well-known technology used in a variety of fields, and thus a detailed description thereof will be omitted.

Meanwhile, the specimen sampling kit identification portion 1611 may identify the specimen sampling kit 500 by receiving the unique ID assigned to the specimen sampling kit 500 or the personal information regarding the person of the corresponding specimen sampling kit 500.

The action tracking portion 1612 is configured to track (or monitor) the action (or motion) of the specimen provider 10 or the carrier handling the specimen sampling kit 500. As a result of the tracking, sampling action information is obtained. The sampling action information includes information regarding what action specimen provider 10 or the carrier is taking and the type of the action.

Here, the action tracked by the action tracking portion 1612 includes a variety of actions. For example, the action includes at least one of a first action of taking the swab from the specimen sampling kit 500, a second action of grasping the taken out swab by the specimen provider 10 of the carrier, a third action of inserting the grasped swab into the nose or the mouth of the specimen provider 10, a fourth action of taking the swab out from the nose or the mouth of the specimen provider 10, a fifth action of putting the swab, taken out in the fourth action, into a receiving vessel included in the specimen sampling kit 500, and a sixth action of sealing the receiving vessel, in which the swab is received, by a sealing means included in the specimen sampling kit 500, but is not limited thereto.

When the swab action is performed to saliva spit by the specimen provider 10, the above-described actions may be changed as follows.
a first action of unpacking the specimen sampling kit;
a second action in which the specimen provider spits saliva into a receiving vessel included in the specimen sampling kit; and
a third action of sealing the receiving vessel

Hereinafter, the principle of the tracking will be described.

First, the action tracking portion 1612 receives the still image or the video image, in which the specimen provider 10 or the carrier handles the specimen sampling kit 500, from the image-capturing part 120.

Afterwards, the action tracking portion 1612 tracks the action of the specimen provider 10 or the carrier from the received information. A variety of technologies may be used in the tracking of the action. Hereinafter, examples of such technologies will be described, but the idea of the present disclosure is not limited thereto.

An example technology that may be used in the action tracking includes a Kinect-based pose recognition algorithm disclosed in Korean Patent No. 10-1784410. Specifically, the action tracking portion 1612 may generate skeleton information containing information regarding the position of the fingertips, elbows, or face of the specimen provider 10 or the carrier and then recognize the type of the action that the specimen provider 10 or the carrier carried out on the basis of the skeleton information. In addition, the action tracking portion 1612 may generate a plurality of pieces of skeleton information over time and connect the plurality of pieces of skeleton information, generated in this manner, in the chronological order. Then, the action may be recognized and tracked on the basis of the connected information.

In this regard, the action tracking portion 1612 may additionally receive three-dimensional (3D) depth information obtained by a depth sensor, in addition to the still image or the video image received from the image-capturing part 120. (In this case, the depth sensor may be a component of the self-sampling management electronic device 100.) The depth information is used to generate the skeleton information.

The Kinect-based pose recognition algorithm is a known technology described in Korean Patent No. 10-1784410, as described above, and a variety of other documents, and thus a further description thereof will be omitted.

Another example technology that may be used in the action tracking includes a neural network-based algorithm for recognizing the action of an individual in a video image disclosed in Korean Patent No. 10-2138680. Here, the neural network may be a convolutional neural network (CNN) subjected to supervised learning on the basis of a deep learning algorithm. Hereinafter, the principle of tracking actions from a video image using the CNN will be described in brief.

First, a video image may be divided into a plurality of frames over time. When the video image is divided, each of the plurality of frames obtained by the division is a still image.

From each of the plurality of divided frames, a first object (e.g., the specimen provider 10 or the carrier) that is the subject of a motion and motion information (i.e., information indicating what motion the first object is taking) regarding the motion of the first object may be extracted. In addition, a second object (e.g., the specimen sampling kit 500) positioned around the first object and positional information of the second object (i.e., information indicating in what portion of the corresponding frame the second object is positioned) may be extracted. Here, the known CNN may be used for the extraction of the objects, the extraction of the motion information, and the extraction of the positional information.

A result of the extraction of the object, the motion information, and the positional information is illustrated in FIG. 5. In FIG. 5, the first object is a person, whereas the second object is a bicycle. Each of the first object and the second object may be extracted using the CNN as described above.

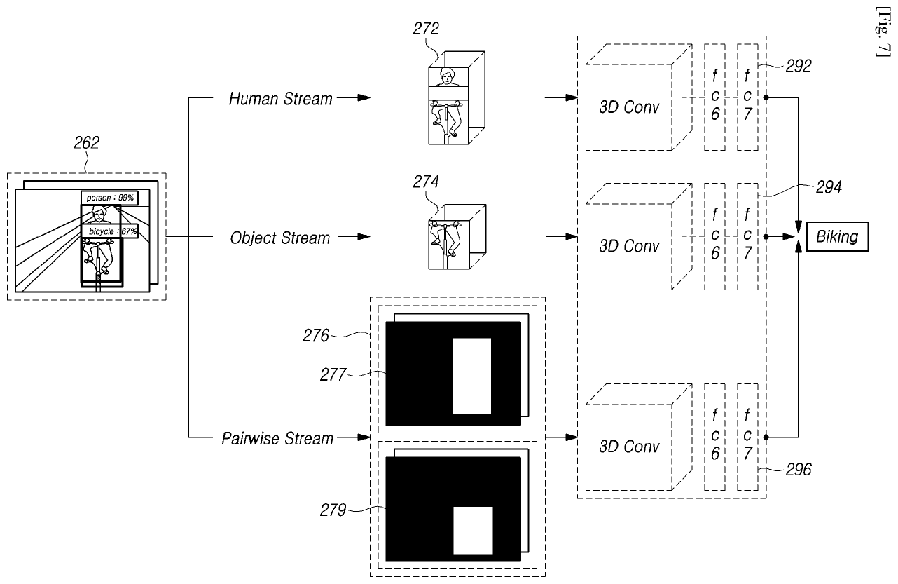

Afterwards, the pieces of motion information regarding the first object extracted from respective frames are connected in the chronological order. That is, a total of N number of pieces of motion information regarding the first object may be extracted by extracting a single piece of motion information from each of N number of frames, and the N number of pieces of motion information are connected in the order of the frames. When connected in this manner, a connection result of the first object is referred to as a first action stream indicating how the motion of the first object has changed in the chronological order. FIG. 6 illustrates the motion of the first object in a plurality of frames.

In addition, the positional information regarding the second object is also connected in the chronological order. In this manner, a connection result of the second object is referred to as a second action stream indicating how the position of the second object has changed in the chronological order.

Furthermore, pieces of information regarding the relative position between the first object and the second object are extracted from the plurality of frames, respectively, and are connected in the chronological order. A result is referred to as a pairwise stream (or combined stream) indicating the relative position between the first object and the second object.

Afterwards, the first action stream, the second action stream, and the pairwise stream are input to an input terminal of the action tracking portion 1612. Then, an inference result regarding what actions the first object has taken in relation to the second object is output from an output terminal of the CNN-based action tracking portion 1612. FIG. 7 illustrates such an example. Referring to FIG. 7, a first action stream 272 regarding the person serving as the first object (i.e., a human stream), a second action stream 274 regarding the bicycle serving as the second object, and the pairwise stream 279 regarding the first object and the second object are input to the input terminal of the CNN. Consequently, as an inference result, the type of the action “riding a bicycle” is output from the output terminal of the CNN.

This technology may be used in the field according to an embodiment in order to track the actions the first object (i.e., the specimen provider 10 or the carrier) having performed in relation to the second object (i.e., the specimen sampling kit 500 or the swab 501).

Details regarding the algorithm for recognizing the actions of the object in the still image or the video image, on the basis of the neural network, in particular, the CNN, are described in Korean Patent No. 10-2138680, and thus, a detailed description of the corresponding algorithm will be omitted herein.

As another example technology that may be used in the action tracking, an algorithm using at least one of the openCV library, a CNN, and a long short-term memory (LSTM) may be used. Here, the LSTM is a neural network subjected to supervised learning on the basis of a deep learning algorithm. In the case of sequential data processing, the LSTM may process data by also considering the sequential order.

Hereinafter, the principle of tracking the action from the video image using the openCV library, CNN, and LSTM will be described in brief.

Frist, the openCV library provides a variety of functions. For example, when the corresponding library is used, it is possible to acquire a video image, the color, size, or resolution of which are changed from those of the original video image. When the video image changed in this manner is used in learning, the CNN or the LSTM robust to, for example, data noise may be obtained.

In addition, when the corresponding library is used, a caption may be overlaid on the video image. According to an embodiment, the caption may illustrate the corresponding video image, i.e., depict what actions the object appearing in the corresponding video image is taking.

Next, the CNN is the same as described above. The CNN may perform object recognition on the plurality of frames of the video image. For example, what object the video image has, what action the object is taking in the corresponding frame, and the like may be inferred by the CNN.

In addition, the LSTM combines object recognition results, which the CNN has inferred for the plurality of frames, in their order. Thus, the action that the object appearing in the video image may be inferred through the plurality of frames. That is, the corresponding video image may be inferred.

These components may be combined to each other as follows to operate in the learning step.

For example, in the learning step, the openCV library changes the color, size, resolution, or the like of the original video image to be used in the learning. Here, in some embodiments, such changes in the video image may be enabled by a technique known as “data augmentation in CNN” instead of the openCV library.

In addition, in the CNN and the LSTM, the original video image and the video image changed by the openCV library are used for the learning. Here, on the basis of output results in the LSTM, the openCV library allows a caption to be overlaid on the video image so as to show the action performed by the object.

In addition, upon completion of the learning, the openCV library, the CNN, and the LSTM may operate as follows. First, the video image from which the action is to be recognized is input to the CNN. Then, the CNN delivers a result of the recognition of the corresponding video image to the LSTM. Here, the result delivered to the LSTM may be a result inferred for each of the frames included in the corresponding video image. In addition, the LSTM infers the action performed by the object appearing in the corresponding video image, on the basis of the result of the inference delivered from the CNN, and outputs a result of the inference. Then, the openCV library generates a caption on the basis of the output result inferred by the LSTM, and operates so that the generated caption is overlaid on the video image.

Here, each of the actions that the openCV library and the corresponding library take may not be performed in some embodiments.

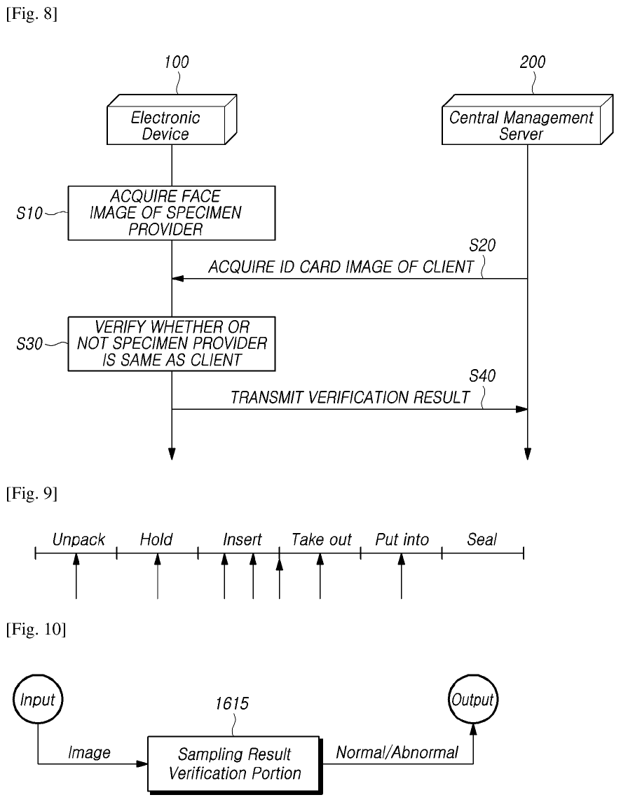

FIG. 3 will be referred to again. The identity verification portion 1613 is configured to verify whether the specimen provider 10 is the same person as the person. Hereinafter, a verification process performed by the identity verification portion 1613 will be described with reference to FIG. 8.

FIG. 8 is a flowchart illustrating a process in which the self-sampling management electronic device 100 verifies whether or not the specimen provider is the same person as the person using the identity verification portion 1613 according to an embodiment. However, FIG. 8 is merely an example, and the idea of the present disclosure will not be interpreted as being limited to the illustration of FIG. 8.

Referring to FIG. 8, in step S10, the self-sampling management electronic device 100 acquires a face image of the specimen provider 10.

Afterwards, in step S20, the identity verification portion 1613 of the self-sampling management electronic device 100 acquires an ID card image of the person. To perform step S20, a following process (not shown in FIG. 8) may be performed, but the present disclosure is not limited thereto.

First, as described above, the specimen sampling kit identification portion 1611 acquires personal information (e.g., name, gender, or address) regarding the person.

Afterwards, the personal information regarding the person is delivered to the central management server 200 through the communication part 110 of the self-sampling management electronic device 100.

Then, the central management server 200 acquires the ID card image of the person on the basis of the personal information regarding the person delivered thereto. In this regard, ID card images of other persons, in addition to the ID card image of the person, may be stored in the database of the central management server 200. Alternatively, the central management server 200 may receive the ID card image of the person by requesting an external verification organization managing ID card images of individuals provide the ID card image of the person. In such a request, the central management server 200 may use the personal information regarding the person delivered from the self-sampling management electronic device 100.

After step S20, in step S30, the identity verification portion 1613 of the self-sampling management electronic device 100 verifies whether or not the specimen provider 10 is the same person as the person. Described in more detail, the identity verification portion 1613 compares the captured image of the specimen provider 10 and the information regarding the person and verifies the identity of the specimen provider 10 on the basis of a result of the comparison.

In this regard, although not shown in the figures, the identity verification portion 1613 may include a feature point extracting portion and a feature point comparing portion. The feature point extracting portion is configured to extract feature points from the face in the captured image. The feature point extracting portion is also configured to extract feature points from the face in ID card image. The feature point comparing portion compares the two types of feature points extracted by the feature point extracting portion, thereby verifying whether or not the specimen provider 10 is the same person as the person. Here, the configurations of the feature point extracting portion and the feature point comparing portion are well known in the art, and thus detailed descriptions thereof will be omitted.

In step S40, a result verified in step S30 is delivered to the central management server 200 of the self-sampling management electronic device 100.

Step S10 and step S40 illustrate identity verification on the basis of the ID card image of the person and the captured face image of the specimen provider 10, but the idea of the present disclosure is not limited thereto. For example, in the identity verification, the following process may be performed separately from or in addition to the above-described process. Hereinafter, the process for the identity verification will be described in detail.

According to an embodiment, the identify may be verified on the basis of the action or the action of the specimen provider 10. Described in detail, first, the person performs his or her own action. This action may include a variety of actions. The person may, for example, move an arm as if drawing a star or raise the right hand and raise the left arm to the side while bowing the waist, but the action of the person is not limited thereto. Then, the self-sampling management electronic device 100 possessed by the person or a particular smartphone records this action. The image captured in the recording is transmitted to the central management server 200. The above-described process of performing the unique action and transmitting the image should have been performed previously.

Subsequently, in a situation in which actual verification should be performed, the self-sampling management electronic device 100 acquiring an image by capturing the action that the specimen provider 10 is taking. This image may be an image captured by the image-capturing part 120 of the self-sampling management electronic device 100.

Afterwards, the identity verification portion 1613 of the self-sampling management electronic device 100 acquires a previously captured image of the person regarding the above-described action. In this regard, as described above, the information (e.g., name, gender, or address) regarding the person acquired by the specimen sampling kit identification portion 1611 may be transmitted to the central management server 200 and, responsively, the previously captured image of the person may be received from the central management server 200.

Then, the identity verification portion 1613 of the self-sampling management electronic device 100 verifies whether or not the specimen provider 10 is the same person as the person. Described in more detail, the identity verification portion 1613 compares the captured image of the specimen provider 10 and the action information in the stored image of the person and performs the identify verification on the basis of a result of the comparison.

In this regard, although not shown in the figures, the identity verification portion 1613 may include an action extracting portion and an action comparing portion. The action extracting portion is configured to extract features from the action of the person in the captured image. The action extracting portion is also configured to extract features from the acquired image of the person. The action comparing portion compares the features of the two types of actions extracted by the action extracting portion and verifies whether or not the specimen provider 10 is the same person as the person on the basis of whether the features of the first action are the same as the features of the second action. Here, the configurations of the action extracting portion and the action comparing portion are well known in the art, and thus detailed descriptions thereof will be omitted.

FIG. 3 will be referred to again. The captured image verified by the identity verification portion 1613 is an image captured after start and before end of the action tracking by the action tracking portion 1612. Hereinafter, points in time at which the image is captured, the number by which the image is captured, and resultant number of verifications will be described as an example.

First, the captured image of the specimen provider 10 may be an image captured while one of the first to sixth actions is being performed.

Here, the selected one action may be, for example, the third action of inserting the specimen sampling kit 500 into the nose or the mouth of the person, but is not limited thereto. When the specimen provider 10 is verified to be the same person as the person on the basis of the image captured during the third action, it may be regarded that the verification has been performed most reliably.

Alternatively, the selected one action may be randomly selected from the six actions.

Second, when the first to sixth actions are sequentially performed in a predetermined order, the captured image of the specimen provider 10 may be an image captured at a “point in time between” adjacent actions, i.e., a point in time after one action ends and before the next action starts.

Here, the selected point in time between adjacent actions may be a point in time between the third and fourth actions, but is not limited thereto. When the specimen provider 10 is verified to be the same person as the person on the basis of the image captured at the point in time between the third and fourth actions, it may be regarded that the verification has been performed most reliably.

Alternatively, the selected point in time between adjacent actions may be a point in time randomly selected among points in time each present between adjacent actions among the six actions.

Third, the captured image of the specimen provider 10 may be an image captured a plurality of times. Here, the plurality of times may be present in at least two adjacent actions among the first to sixth actions. In addition, the identity verification portion 1613 may verify the identity on the basis of respective pieces of image captured the plurality of times.

In addition, the points in time at which the image of the specimen provider 10 is captured are indicated with arrows in FIG. 9. Referring to FIG. 9, each point in time at which the image is captured may be one or more points in time during the first action related to the unpacking, the second action related to the grasping, the third action related to the insertion, the fourth action related to the taking out of the swab, the fifth action related to the reception of the swab, and the sixth action related to the sealing of the receiving vessel or one or more points in time each present between adjacent action among the six actions.

FIG. 3 will be referred to again. The action evaluation portion 1614 is configured to evaluate the sampling action information acquired by the tracking of the action tracking portion 1612.

Described in detail, reference operation information required for the sampling is stored in the action evaluation portion 1614.

The reference sampling action information contains types of actions to be performed during the sampling, i.e., information regarding the first to sixth actions, and information regarding the order of the first to sixth actions.

In addition, the reference sampling action information contains reference information regarding the manner in which each of the first to sixth actions is to be performed. For example, the reference information regarding a depth of insertion (hereinafter, referred to as a “reference depth of insertion”) and information regarding an angle of insertion (hereinafter, referred to as a “reference angle of insertion”) required when, for example, the swab of the specimen sampling kit 500 is inserted into the nose or the mouth of the specimen provider 10. Here, the reference angle of insertion may refer to a relative angle of the swab grasped by the specimen provider 10 or the carrier with respect to the longitudinal direction of the nose or the direction facing into the mouth. In addition, a reference length of insertion may refer to the length of a portion of the swab not inserted when the specimen provider 10 or the carrier has inserted a portion of the swab into the nose or the mouth of the specimen provider 10 by grasping the swab.

The action evaluation portion 1614 compares the reference sampling action information with the sampling action information acquired by the tracking of the action tracking portion 1612.

For example, the action evaluation portion 1614 evaluates whether or not each of the first to sixth actions is included in the sampling action information, i.e., whether or not any of the actions is missing.

Alternatively, the action evaluation portion 1614 evaluates whether or not feature information included in the sampling action information is the same as feature information included in the corresponding reference sampling action information and, if not the same, the difference between the two pieces of feature information. When the difference is greater than a threshold, the action evaluation portion 1614 evaluates that the action corresponding to the difference greater than the threshold has not been properly performed.

Here, the evaluation technology of comparing the sampling action information and the reference sampling action information by the action evaluation portion 1614 is well known in the art, and thus a detailed description thereof will be omitted.

The sampling result verification portion 1615 is configured to verify whether or not the specimen is properly sampled by the swab of the specimen sampling kit 500.

In this regard, the sampling result verification portion 1615 receives an image of the swab, by which the specimen has been sampled, from a data acquiring portion. In addition, the sampling result verification portion 1615 verifies whether or not the specimen has been properly sampled and outputs a result “normal” or “abnormal” on the basis of the image of the swab received in this manner. The sampling result verification portion 1615 is schematically illustrated in FIG. 10.

Meanwhile, the sampling result verification portion 1615 may include a model realized by the CNN. Hereinafter, a learning method of this model will be described in detail.

First, the CNN may be learned by a learning device (not shown) by supervised learning. In addition, the learning device includes a processor, such as a graphics processing unit (GPU), but is not limited thereto.

The learning process will be described. First, learning data is prepared. The learning data is categorized into learning input data and learning answer data. The learning input data includes a plurality of images. A sampling-completed swab (i.e., a swab by which the sampling is completed) is depicted on each of the images. In addition, the learning answer data includes “normal” and “abnormal” as answers as to whether or not the sampling has been properly performed using the swab depicted on each image.

The learning data may be prepared in a variety of methods. For example, the learning data may be obtained by performing sampling normally on some of a plurality of swabs and abnormally on the remaining of the plurality of swabs and capturing images of results of the sampling. Afterwards, answers are assigned to the acquired images.

Here, the swab of abnormal sampling may mean, for example, a situation in which a sufficient amount of specimen is not attached to the swab or a situation in which a specimen is attached to a portion of the swab rather than a portion of the swab to which the specimen is supposed to be attached. On the captured image, the swab of abnormal sampling may differ from a swab of normal sampling.

Afterwards, the plurality of pieces of learning input data are input to the CNN and are compared with inferred output data of the CNN. In this process, differences between the inferred output data and the learning answer data, i.e., errors, are delivered to a recurrent neural network by backpropagation. Consequently, parameters included in the recurrent neural network, for example, weights and bias values are updated so that such errors may be minimized. The backpropagation is a well-known technology, and thus a detailed description thereof will be omitted.

The learning is performed until the performance of the CNN meets a predetermined condition. The predetermined condition may be determined by a variety of methods. For example, the predetermined condition may be determined by cross-validation. Regarding the cross-validation, a portion of the learning data is assigned as validation data. In the learning process, not only the learning data but also the validation data is used. For example, the degree of error regarding a result of use of the learning data is checked, and the degree of error regarding a result of use of the validation data is also checked from time to time during the process. In a situation in which the degree of error of the validation data has a minimum value even though the degree of error of the learning data is decreasing, the condition is met at that point in time.

FIG. 3 will be referred to again. The measure generating portion 1616 is configured to generate a variety of measures. The variety of measures may be generated on the basis of at least one of the test result performed by the identity verification portion 1613, the evaluation result performed by the action evaluation portion 1614, and the test result performed by the sampling result verification portion 1615, but are not limited thereto.

The measures will be described in detail, and the following message types are illustrative only.

First, the measure generating portion 1616 may generate a message on the basis of the test result performed by the identity verification portion 1613. The message includes information regarding whether or not the specimen provider 10 is the same person as the person in the sampling process. For example, when the specimen provider 10 in the sampling process is “A” but the person is not “A” according to the result of the verification, the measure generating portion 1616 may generate a message stating that “the specimen provider is not the same as the person.” The message generated in this manner may be displayed on the display part 150 of the self-sampling management electronic device 100 or output through the speaker 140 of the self-sampling management electronic device 100. In addition, the message generated in this manner may be transmitted to the central management server 200 or the terminal of the diagnosis center 300 through the communication part 110 of the self-sampling management electronic device 100.

Second, the measure generating portion 1616 may generate a message on the basis of the evaluation result performed by the action evaluation portion 1614.

The message generated in this manner may include information regarding any missing action among the first to sixth actions. In this case, the message may further include an instruction stating that the missing action should be performed.

Alternatively, the message may include, for example, information regarding whether or not each of the first to sixth actions has been performed in compliance with the predetermined condition. For example, the message may include information regarding whether or not the depth of insertion or the angle of insertion measured from the image is the same as the reference depth of insertion or the reference angle of insertion when the swab is inserted into the nose or the mouth of the specimen provider 10 in the third action. When the depths or the angles are not the same, a message stating “the depths or the angles should be the same” may be included. (Reference numeral 152 in FIG. 1 indicates an example of this message.)

As described above, according to an embodiment, it is possible to verify whether the person who provided the specimen in the sampling process, i.e., the specimen provider 10, is the same person as the person.

In addition, an evaluation or a feedback regarding the actions related to the sampling may be delivered to the person who has performed the sampling or the diagnosis center managing the sampling, thereby verifying whether or not the sampling has been properly performed.

In addition, the still image or the video image captured by the image-capturing part 120, the personal information acquired by the specimen sampling kit identification portion 1611, or the like may be deleted by the processor 170 when at least one of the tracking process by the action tracking portion 1612, the identity verification process by the identity verification portion 1613, the action evaluation process by the action evaluation portion 1614, and the sampling result verification process by the sampling result verification portion 1615. Consequently, it is possible to prevent the leakage of the still image, the video image, or the personal information.

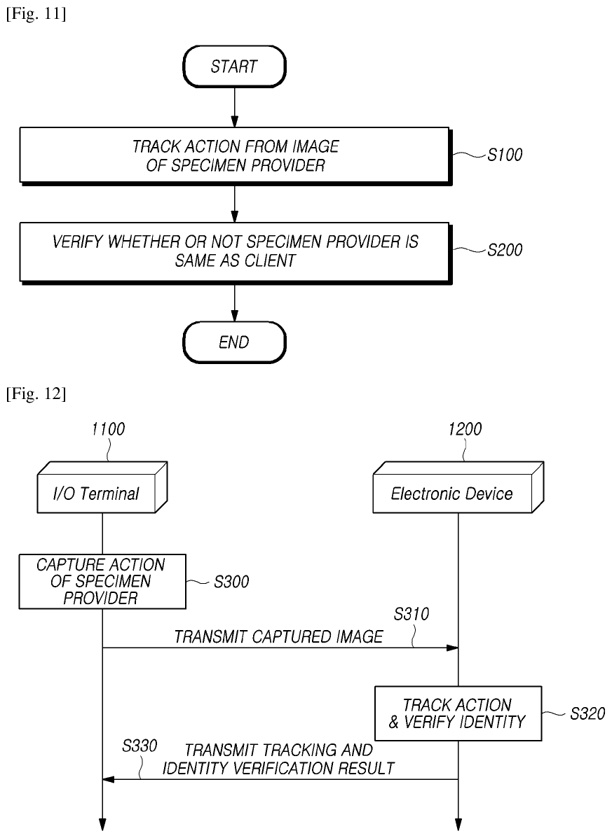

FIG. 11 is a flowchart illustrating procedures of a self-sampling management method according to an embodiment. However, FIG. 11 is illustrative only, and the idea of the present disclosure will not be interpreted as being limited to the flowchart illustrated in FIG. 11. For example, the self-sampling management method may be performed without at least one of the steps illustrated in FIG. 1, may further include a step not illustrated in FIG. 1, or may be performed in an order different from that illustrated in FIG. 1.

Referring to FIG. 11, step S100 of tracking the action of the specimen provider 10 from the image of the specimen provider 10 handling the specimen sampling kit 500 is performed.

In addition, step S200 of verifying whether or not the specimen provider 10 is the same person as the person of the specimen sampling kit 500 is performed, on the basis of an image captured after start and before end of the tracking among the image of the specimen provider 10 handling the specimen sampling kit 500.

In addition, the self-sampling management method is performed by the self-sampling management electronic device 100 illustrated in FIG. 3. For a detailed description of the self-sampling management method, the description of the self-sampling management electronic device 100 will be referred to.

In addition, the self-sampling management electronic device 100 may be a portable communication terminal, a smartphone, a wearable device, a tablet PC, a desktop PC, a laptop computer, or the like, as described above, or a kiosk. Hereinafter, an embodiment in which the self-sampling management electronic device 100 is a kiosk or the like will be described.

When the self-sampling management electronic device 100 is a kiosk or the like, the self-sampling management electronic device 100 is positioned in a public place, such as a station or an airport.

Thus, the specimen provider 10 visits such a public place to use the self-sampling management electronic device 100.

The self-sampling management electronic device 100 reviews the identity of the specimen provider 10 by receiving an ID card or the like of the specimen provider 10.

Afterwards, the self-sampling management electronic device 100 discharges the specimen sampling kit 500 exclusively for the specimen provider 10. In this regard, the self-sampling management electronic device 100 may include a storage part in which a plurality of specimen sampling kits 500 are stored and a discharge port through which the specimen sampling kits 500 are discharged.

The specimen provider 10 receives and unpacks the discharged specimen sampling kit 500, samples his or her own specimen using the specimen sampling kit 500, and seals the specimen sampling kit 500. The sealed specimen sampling kit 500 is collected by the self-sampling management electronic device 100. In this regard, the self-sampling management electronic device 100 is provided with a collection part to collect the specimen sampling kits 500.

Here, the actions of the specimen provider 10, started by receiving the discharged specimen sampling kit 500 and unpacking the discharged specimen sampling kit 500 to sample his or her own specimen and finished by sealing the specimen sampling kit 500, are tracked by the self-sampling management electronic device 100 illustrated in FIG. 3. Furthermore, on the basis of the image of the specimen provider 10 captured during the tracking process, whether or not the specimen provider 10 is substituted by another person, i.e., the specimen provider 10 is the same as the initially-identified person, is verified.

In this regard, the specimen provider 10 is required to perform the process of sampling his or her own specimen in front of the image-capturing part 120 of the self-sampling management electronic device 100. In some embodiments, the display part 150 of the self-sampling management electronic device 100 may display a message stating that the specimen provider 10 should be positioned in front of the image-capturing part 120 of the self-sampling management electronic device 100 in the sampling of the specimen.

In addition, the self-sampling management electronic device 100 may be implemented as a separate server. This will be described hereinafter with reference to FIG. 12.

FIG. 12 is a flowchart illustrating an information delivery process between an input/output (I/O) terminal 1100 and the self-sampling management electronic device 1200 when the self-sampling management electronic device 1200 is a separate server. However, FIG. 12 is illustrative only, and the idea of the present disclosure will not be interpreted as being limited to the illustration of FIG. 12.

Referring to FIG. 12, the I/O terminal 1100 refers to a terminal configured to receive or output information for the specimen provider 10. The I/O terminal 1100 may include terminals, such as a smartphone, a smart pad, and a personal digital assistant (PDA), but is not limited thereto.

For example, in step S300, the image of the action of the specimen provider 10 is acquired in the I/O terminal 1100. The image acquired in this manner may be an image captured by the I/O terminal 1100.

In step S310, the captured image is delivered to the self-sampling management electronic device 1200 through the network 600.

In step S320, the self-sampling management electronic device 1200 verifies whether or not the specimen provider 10 is the person on the basis of the image delivered in this manner and verifies whether or not the sampling action has been properly performed. These actions and preceding and subsequent actions are the same as those performed by the self-sampling management electronic device 100 described above with reference to FIG. 3.

In step S330, a result of the verification in step S320 is transmitted to the I/O terminal 1100 by the self-sampling management electronic device 1200.

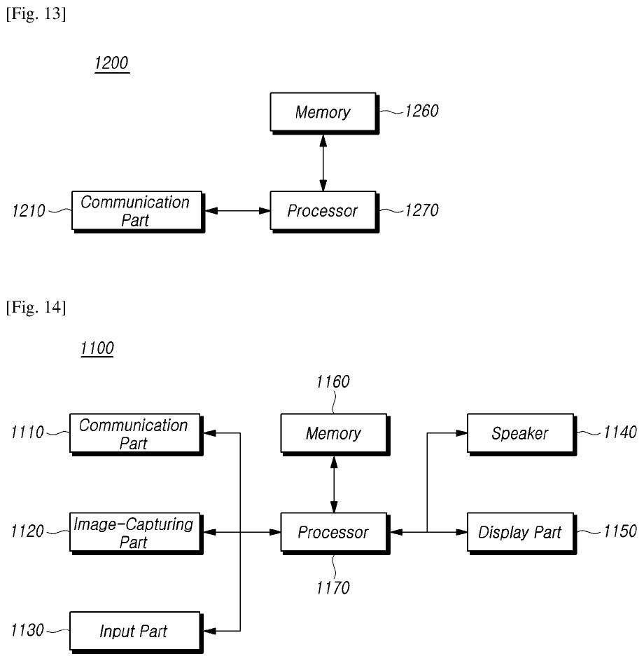

In this regard, the self-sampling management electronic device 1200 may have a configuration illustrated in FIG. 13. Referring to FIG. 13, the self-sampling management electronic device 1200 includes a communication part 1210, a memory 1260, and a processor 1270. Here, the communication part 1210, the memory 1260, and the processor 1270 have the same functions as the communication part 110, the memory 160, and the processor 170 illustrated in FIG. 3, respectively. Thus, for descriptions of the communication part 1210, the memory 1260, and the processor 1270, the descriptions of the communication part 110, the memory 160, and the processor 170 with reference to FIG. 3 will be referred to.

Furthermore, in this regard, the I/O terminal 1100 may include a configuration illustrated in FIG. 14. Referring to FIG. 14, the I/O terminal 1100 includes a communication part 1110, an image-capturing part 1120, an input part 1130, a speaker 1140, a display part 1150, a memory 1160, and a processor 1170, but is not limited thereto. For example, the self-sampling management electronic device 100 may not include at least one of the components illustrated in FIG. 14 or further include any component not illustrated in FIG. 14.

Here, the communication part 1110, the image-capturing part 1120, the input part 1130, the speaker 1140, and the display part 1150 are the same as the components illustrated in FIG. 3, i.e., the communication part 110, the image-capturing part 120, the input part 130, the speaker 140, and the display part 150. For example, the image-capturing part 1120 captures an image of the specimen provider handling a specimen sampling kit. The communication part 1110 transmits the acquired image to the electronic device 1200, and responsively, receives a test result as to whether the specimen provider 10 is the same person as the person who requested the specimen sampling kit 500. The display part 1150 displays the received test result.

Furthermore, the memory 1160 stores instructions, a program, or the like by which the actions are performed. In addition, the processor 1170 operates to perform predetermined functions by executing the instructions or program stored in the memory 1160.

The components 1110 to 1170 match the components 110 to 170 illustrated in FIG. 3, respectively. Thus, for descriptions of the components 1110 to 1170, the above descriptions of the components 110 to 170 with reference to FIG. 3 will be referred to.

In addition, according to another embodiment, a self-sampling management method performed by the self-sampling management electronic device may include steps of: tracking the sampling action of the specimen provider from the image of the specimen provider handling the specimen sampling kit; comparing the tracked sampling action with a reference action required for the sampling; and generating contents to be provided to the specimen provider on the basis a result of the comparison. Furthermore, this method may be performed by the self-sampling management electronic device, and respective steps of this method may be implemented as a computer program stored in a computer readable recording medium.

In addition, each of the foregoing methods according to a variety of embodiments may be implemented as a computer program configured to perform the steps of the method and stored in a computer readable recording medium. In addition, each of the methods may be implemented as a computer readable recording medium storing a computer program configured to perform the steps of the method.

The above description provides an example of the technical idea of the present disclosure for illustrative purposes only. Those having ordinary knowledge in the technical field, to which the present disclosure pertains, will appreciate that various modifications and changes in form, such as combination, separation, substitution, and change of a configuration, are possible without departing from the essential features of the present disclosure. Therefore, the embodiments disclosed in the present disclosure are intended to illustrate the scope of the technical idea of the present disclosure, and the scope of the present disclosure is not limited by the embodiments. The scope of the present disclosure shall be construed on the basis of the accompanying claims in such a manner that all of the technical ideas included within the scope equivalent to the claims belong to the present disclosure.

### CROSS-REFERENCE TO RELATED APPLICATIONS

This application claims priority from Korean Patent Application No. 10-2021-0052579, filed on Apr. 22, 2021 in the Korean Intellectual Property Office, the disclosure of which is incorporated herein by reference in its entirety.

1. A self-sampling management electronic device comprising:
a memory storing instructions; and
at least one processor,
wherein the processor is configured, by executing the instructions, to:
track a sampling action of a specimen provider by using images of the specimen provider handling a specimen sampling kit; and
verify whether or not the specimen provider is the same person as a person who has requested the specimen sampling kit, by using at least one of the images used for the tracking.

2. The self-sampling management electronic device of claim 1, wherein the processor is configured, by executing the instructions, to:
compare the tracked sampling action information with reference sampling action information to be followed by the specimen provider when the specimen provider performs the sampling action; and
generate contents to be provided to the specimen provider on basis of a result of the comparison.

3. The self-sampling management electronic device of claim 2, wherein the reference sampling action information comprises information regarding a reference depth of insertion and/or a reference angle of insertion required when a swab of the specimen sampling kit is inserted into a nose or a mouth of the specimen provider, and the tracked sampling action information comprises information regarding a measured depth of insertion and/or a measured angle of insertion measured from an image of the swab inserted into the nose or the mouth of the specimen provider.

4. The self-sampling management electronic device of claim 1, wherein the tracked sampling action comprises at least one of:
a first action of unpacking the specimen sampling kit;
a second action of grasping a swab included in the unpacked specimen sampling kit;
a third action of inserting the grasped swab into a nose or a mouth of the specimen provider;
a fourth action of taking the swab out from the nose or the mouth of the specimen provider;
a fifth action of putting the swab into a receiving vessel included in the specimen sampling kit; and
a sixth action of sealing the receiving vessel in which the swab is received.

5. The self-sampling management electronic device of claim 1, wherein the tracked sampling action comprises at least one of:
a first action of unpacking the specimen sampling kit;
a second action in which the specimen provider spits saliva into a receiving vessel included in the specimen sampling kit; and
a third action of sealing the receiving vessel.

6. The self-sampling management electronic device of claim 4, wherein the processor is configured, by executing the instructions, to:
verify whether or not the tracked sampling action includes all the sampling actions; and
generate contents to be provided to the specimen provider on basis of a result of the verification.

7. The self-sampling management electronic device of claim 4, wherein the image for the verification comprises an image obtained from the third action.

8. The self-sampling management electronic device of claim 7, wherein the image for the verification comprises an image randomly selected from the images obtained from the first to the sixth actions.

9. The self-sampling management electronic device of claim 4, wherein the image for the verification comprises an image obtained from at least two adjacent actions among the first to sixth actions.

10. The self-sampling management electronic device of claim 1, wherein the processor is configured, by performing the instructions, to generate a message to be displayed to the specimen provider or a message to be transmitted to a terminal of a diagnosis center managing the specimen sampling kit when the specimen provider is not the same person as the person who has requested the specimen sampling kit.

11. The self-sampling management electronic device of claim 1,
wherein the sampling action is performed on the specimen provider by a delivery person who has delivered the specimen sampling kit to the specimen provider, and
the images used for the tracking are obtained from the sampling action performed by the delivery person.

12. A self-sampling management method performed by a self-sampling management electronic device, the method comprising:
tracking a sampling action of a specimen provider by using images of the specimen provider handling a specimen sampling kit; and
verifying whether or not the specimen provider is the same person as a person who has requested the specimen sampling kit, by using at least one of the images used for the tracking.

13. A computer readable recording medium storing a computer program configured to perform operations included in the method of claim 12.

14. A computer program stored in a computer readable recording medium configured to perform operations included in the method of claim 12.

15. A self-sampling management method performed by a self-sampling management electronic device, the method comprising:
tracking a sampling action of a specimen provider by using images of the specimen provider handling a specimen sampling kit;
comparing the tracked sampling action with a reference action to be followed by the specimen provider when the specimen performs the sampling action; and
generating contents to be provided to the specimen provider on basis of a result of the comparison.

16. The self-sampling management electronic device of claim 5, wherein the processor is configured, by executing the instructions, to:
verify whether or not the tracked sampling action includes all the sampling actions; and
generate contents to be provided to the specimen provider on basis of a result of the verification.
